(19) United States
(12) Patent Application Publication
(10) Pub. No.: US 2010/0081921 A1
(43) Pub. Date: Apr. 1, 2010

(54) METHOD FOR UPDATING A STATUS OF A MEDICALLY USABLE OBJECT

(76) Inventors: Alexander Urban, Forstinning (DE); Ingmar Thiemann, Munchen (DE)

Correspondence Address: DON W. BULSON (BRAI), RENNER, OTTO, BOISSELLE & SKLAR, LLP, 1621 EUCLID AVENUE - 19TH FLOOR, CLEVELAND, OH, 44115, US

(21) Appl. No.: 12/562,192

(22) Filed: Sep. 18, 2009

Related U.S. Application Data
(60) Provisional application No. 61/121,930, filed on Dec. 12, 2008.

(30) Foreign Application Priority Data
Sep. 29, 2008 (EP) 08165412

(51) Int. Cl. A61B 5/05

(52) U.S. Cl. 600/424

(57) ABSTRACT

The present application relates to a medical data method for updating a status of at least one object which relates to a medical applicability of the at least one object, including the following steps: preparing object data which describes location data and the status of the at least one object; preparing change data which relates to changes in the status of the at least one object; wherein the status of the at least one object is updated on the basis of a comparison between the object data and the change data.

### RELATED APPLICATION DATA

This application claims the priority of U.S. Provisional Application No. 61/121,930, filed on Dec. 12, 2008, which is hereby incorporated in its entirety by reference.

### FIELD OF THE INVENTION

The present invention relates to a medical data method for updating a status of at least one medically usable object.

### BACKGROUND OF THE INVENTION

In practical work in the field of medicine, care must in most cases be taken that the working environment and the implements fulfill particular criteria. In this respect, it can for example be necessary to determine whether a work surface, a space, the surface of the body or the medical staff's clothing, a medical instrument, parts of the patient's body or also substances which are to be introduced into the patient's body and/or used on the patient's body fulfill requirements regarding intactness, cleanness and sterility. In the case of operations, it is also necessary to be clear on where surgical implements are after the end of the operation, in order for example to prevent an instrument from remaining in the patient's body after the operation and causing damage to the patient's body. For the management of a modern medical facility, it is also important to know how its stock-keeping has to be conducted in order to operate economically.

The subject of the present invention is to provide a method, with the aid of which the spatial location and/or the movement of an object can be determined and/or tracked, wherein the object can include a medical instrument, a body or body part of a member of the medical staff and/or a patient, and/or a medically usable object. The spatial location of an object can be described by the location data, wherein the location data conceptually describes the location of the object in a global coordinate system or a coordinate system which is defined relative to the location of the object. The location data can also describe the orientation of the object in a global coordinate system or a coordinate system which is defined relative to the location of the object, and/or the orientation of the object relative to the location data of another object which is specified in a global coordinate system or a coordinate system which is defined relative to the location of the first object. The location data can also comprise a movement vector which designates the speed of an object in terms of its magnitude and direction. The term “medically usable object” can include a medical instrument, a swab, a compress, bandaging material, a flexible tube which can be suitable for guiding gas and/or liquid, a catheter, a storage table, a patient couch, a patient bed and more such like objects. An object can also be understood to mean a two-dimensionally or three-dimensionally defined spatial zone which in particular contains a medically usable object and the periphery of which is in particular spaced apart from the object (for example, a patient couch), so as to for example ensure a safety distance from sterile objects. The method in accordance with the invention can simultaneously identify the object, i.e. assign it to a particular type and/or class of objects (such as for example assigning a surgical pair of scissors to the class of medical instruments). The method is also capable of detecting a status of the object; the object is in this respect assigned status data. This includes for example establishing whether the object is clean or not and/or sterile or not and/or undamaged or damaged and/or whether it is currently in use or is unused or whether it has been used. The status can also provide a description as to whether the object is at rest or in motion. An object can also be assigned a plurality of statuses, i.e. in particular more than one status, i.e. also for example two or three or more statuses: a sterile object can for example be currently in use, hence it can be assigned the two corresponding statuses “current in use” and “sterile”. The method is also intended to enable the management of a medical facility to optimize its stock-keeping, by enabling a dataset relating to an object to be provided with information, via the assignment of statuses to the object, for example in a database, which for example specifies whether the object is available in a used or unused form. In such a case, the object preferably comprises a medical utility and/or consumable object. It is then for example possible to deduce whether it is for example necessary to reorder a new object of the same type from the supplier. An object can also comprise a spatial zone, hence the location data of spatial zones can also be determined and/or tracked. These zones can be zones which delineate a space and/or an area and/or plane, which has a particular status, with their periphery (zone periphery). Such a zone can be defined such that it is stationary or also such that it can be moved. If the zone is movable, i.e. can change its location data, then its location data can be defined relative to location data of another object. Changes in status, such as have been envisaged for an object, are possible for a zone within the framework of this invention. Thus, a zone can also for example be provided with a “sterile” status which is updated to the “not sterile” status. The terms “changing a status” and “updating a status” and “assigning a status” are used mutually exchangeably within the framework of this invention.

US 2007/0018810 shows a system and method for radio frequency identification (RFID) and for correspondingly marking implantable medical apparatuses. This document discloses a system consisting of an RFID transmitter/receiver and a transponder which can also be used as a component of a patient management system, in order to observe the course of chronic diseases.

WO 03001329 shows a data processing system for medical apparatuses which uses information on the medical apparatus such as for example a serial number or usage data of the medical apparatus, can manage patient data and accounting data and comprises an interface to the stock-keeping and maintenance database.

WO 2006045080 shows a system for stock-keeping in the field of medicine, which can be implemented inter alia using RFID technology.

WO 200153919 shows a system and method for tracking medical waste, which is based on marking means of transport, for example using optically readable adhesive labels.

### SUMMARY OF THE INVENTION

It is an object of the present invention to provide a method for updating a status of at least one object, which describes a medical applicability of the at least one object.

This object is solved by the subjects of the independent claims. The dependent claims relate to advantageous embodiments of the invention. The features of advantageous embodiments can be combined from among the different embodiments.

The medical data method in accordance with the invention for updating a status of at least one object, which relates to and in particular describes a medical applicability (characteristic) of the at least one object, advantageously includes the following steps: data which relates to the at least one object is prepared, wherein said object data describes location data and the status of the at least one object.

The location data of the object can be understood to mean a description of the location of the object, for example with the aid of multi-dimensional coordinates (for example, Cartesian coordinates, polar coordinates and/or spherical coordinates) and/or vectors (which indicate the orientation of the object by their direction with respect to a reference location and a distance from said reference location). The location data of the object can be specified in a global coordinate system or also relative to another object which can for example differ from the first object. The location data is for example captured, for example by using a camera (i.e. a detection device) which detects the object with the aid of electromagnetic radiation (in particular light in the infrared spectral range) which is reflected or emitted by a marker device attached to the object and transmits detection-based data, in particular to a data processing device which then ascertains the coordinates and/or location data of the object. It is however also possible to attach radio frequency identification (RFID) tags to the object, such that the location data of the object can be ascertained via a data processing device with the aid of an RFID sensor and/or sensors. Other examples, such as for example optically recognizing the shape and location of the object, are mentioned below.

The status of the object relates to—in particular, describes—for example its applicability (characteristic), i.e. it includes information for example with respect to the sterility and/or non-sterility of the object, the intactness of or damage to (deformation of) the object, the cleanness and/or dirtiness of the object, the use and/or non-use of the object, for example whether it is currently in use (i.e. for example, whether it is in direct and/or indirect contact, for example physical contact, with a user and/or whether the user is situated in the zone), has been used or has not yet been used. In this context, “use” means at least that the object and/or zone has been used in a way in which the use was intended. In this way, the position of the object in the workflow can be described, and the object which is to be used next in the workflow can for example be ascertained. On the basis of this ascertainment, it is possible to indicate to the user which object is to be used next. The three aforesaid usage statuses can in particular be determined via the movement history of the object. If the object has not yet been moved (as of a particular start time, which can for example correspond to the beginning of an operation), then the object is determined to be “not yet used”. If it has already been moved, then it is determined to be “already used”. If the object is currently being moved or has been recently moved (for example, within the last 10 seconds or 60 seconds), then it is determined to be “currently in use”. Determining the usage status of the object can alternatively or additionally also be based on other conditions, in particular whether or not the object has come into contact with another object (see below). If an object, for example a medical instrument, comes into contact with another object (for example, the physician), then it is then possible to deduce the current use of the object. This determination that the object is “currently in use” can additionally be made dependent on the movement of the object. The method or system in accordance with the invention can thus determine that a particular stage (step) in the workflow has been reached which corresponds to the use of this instrument. Notifying information (for example, screen and/or audio) can then be correspondingly output to the surgeon as to which step in the procedure was planned next and/or what is planned for the current step. This can in particular be used when registering objects in a number of steps within the framework of image-guided surgery (IGS). Control of an apparatus, for example the lighting control in the operating theatre, can also be based on the particular use, and the instrument currently in use and/or the instrument to be used next (lying for example on a table) can in particular be illuminated more brightly (than other objects).

The invention thus allows the sequences of statuses of a multitude of objects to be monitored. An object can for example transition from a “not yet used” status to an “in use” status and finally to an “already used” status. Another object can then transition from a “not yet used” status to the “currently in use” status and, after a period of use, to the “already used” status. This sequence of statuses can be determined for a multitude of objects. This sequence of statuses can in particular also be planned for a multitude of objects. Preferably, sequence-of-status data is therefore prepared which represents a planned sequence of statuses, for example for a particular operation. This planned sequence of statuses is then compared in accordance with the invention with the determined statuses and thus with the determined sequence of statuses for a multitude of objects. Notifying information can in particular be output on the basis of the comparison. The physician's attention can for example be drawn to the object (a physical object, for example an instrument) which is planned for use next. Warning notifications can also be given if an object other than the planned object is used by the physician. It is thus for example possible to notify the physician if he uses a type of instrument (for example, a type of scissors) which does not correspond to the planned type of instrument (the planned type of scissors). In this way, it is in particular also possible to monitor, in particular at the end of an operation and/or treatment, whether all the physical objects have been removed (such as for example swabs from the patient's body). For example, a swab has the “currently in use” status as long as it is still situated in the zone which surrounds the patient and/or at least a part of the patient's body (such as for example an organ and/or a part of an organ). It is possible to predetermine, as a planned sequence of statuses, that the status of the swab transitions from “currently in use” to “already used” if the swab leaves the patient zone. It can be defined as not being compatible with the planned sequence of statuses if the swab still has the “currently in use” status even though the status of the needle planned for sewing up transitions from “not yet used” to “currently in use”. In this case, a lack of correspondence between the planned sequence of statuses and the current and/or determined sequence of statuses of advantageously different objects (in particular physical objects) is then established, and a warning signal can be output. The warning signal represents an example of notifying information which is output on the basis of the comparison between the planned sequence of statuses and the determined sequence of statuses of advantageously different objects.

Using the method in accordance with the invention, it is also possible to provide a balance of account of objects, in particular physical objects. This means that a number of objects can be established and compared with a comparative number of objects, wherein on the basis of this comparison, information and/or a notification can be output to a user. Information which includes the number of objects and/or the comparative number of objects can in particular be added and/or linked to the status of the object. Information can also be added and/or linked to the status which provides details on whether the object is supposed and/or not in fact supposed to be situated in a particular zone (for example, the zone in which the location of the object has been established). It is also possible to add and/or link information to the status of an object which provides details on the type and/or class of the object and/or whether and/or how many other objects of another type and/or class and/or of the same type and/or class of objects are situated and/or are not in fact situated in a particular zone, in particular at the same time as the object being considered. Adding and/or linking the number of objects to the status of the object is possible within the framework of said method, but not necessary in order to ensure that the method can be performed. At the beginning of an operation, a predetermined number of physical objects can for example be stored in a data processing device. This number (the initial number) can also advantageously be defined by being input by the user via an input module of the data processing device. The user can also indicate the beginning of an operation to the data processing device by an input. The initial number can for example be included in change data. The physical objects can then be detected by the detection device, wherein an initial number of the physical objects is ascertained, wherein the physical objects can be ascertained in terms of their initial number for each type and/or class of a physical object (for example for each type of a particular pair of surgical scissors), and this initial number can be stored in a data processing device. At a point in time which indicates the end of an operation, an end number of the physical objects (which can also be respectively broken down separately according to the type and/or class of the physical objects) is then in turn ascertained and stored in the data processing device. A processor in the data processing device then compares the initial number with the end number. The end number can thus be a comparative number. If this comparison reveals an undesirable deviation between the end number and the initial number, for example an end number which is lower than the initial number, then a corresponding notification and/or information can be output to the user. It is possible, in the step of updating the object, to enter information in the status which provides details on the comparison result. This notification can be made in visual form using an indicating device (for example, a monitor) of the data processing device, or can also include audio information (for example, from a loudspeaker). In particular, the user can be notified that a particular number of physical objects could not be detected at the end time of the operation, i.e. at the point in time at which the end number was ascertained. Depending on where these objects were last localized within the framework of the method, i.e. where their location was last established, visual and/or audio information can be output to the user as to where the physical object was last situated. This serves to make it easier for the user to locate such undetected physical objects. In addition, it is possible to output information to the user as to when the location of the physical object was last situated in a zone which is assigned to at least one part of the patient's body (in particular the operating environment and/or a patient zone, i.e. a zone in which the patient is situated). Such a comparison between the number of detected physical objects and an initial number of physical objects can also be made between the start time and end time of an operation, in order for example to prevent objects which are still required from not being ready for use (i.e. for example, not within the user's reach). It is thus possible to ensure that the required number of particular physical objects is always available. Such a number of physical objects, established between the start time and end time, can also be referred to as an intermediate number, wherein the intermediate number can be used as a comparative number.

The initial number and/or end number can also be ascertained in zones, i.e. for each zone, wherein the number of physical objects is respectively ascertained for a particular zone. This ascertainment of the initial number and end number can in turn simultaneously be broken down according to the type and/or class of the physical objects. It is thus possible to ensure that only particular objects are situated in the zone in question, the presence of which in said zone is also desirable and/or desirable at a particular point in time in the method sequence. It is also possible to ascertain that physical objects which should be situated in the zone are not in fact situated in the zone. Physical objects and their desirable/undesirable and actual presence and/or absence in the zone can be ascertained in this way for each zone by comparing the ascertained number and type of physical objects in the respective zone with a list stored in the data processing device which includes information on which objects are supposed and/or not supposed to be situated in which zone and in which numbers. This list can for example be included in the change data. This requirement regarding the presence and/or absence of a physical object in a particular zone can be dependent on the method sequence and/or on at least one of the start time, intermediate time and end time. It is for example possible to define that a swab is to be situated in a zone which encompasses an instrument table at the start time, but is for example always to be still situated on the instrument table or situated in a disposal container at the end time. A notification corresponding to the comparison can be output to the user in visual form and/or as audio information. It is also possible, in the updating step for the status of the object, to enter information in the status which provides details on the comparison result. This can for example be achieved via a monitor and/or a loudspeaker which is connected to the data processing device. An example of a physical object which should be situated in a particular zone would be particular types of instruments, each in a particular number, on an instrument table. An example of an object which is not supposed to be situated in a particular zone would be a swab which, at the defined end time of the operation, is still situated in a zone which encompasses and/or includes the operating environment. This can for example occur when the operating environment has already been closed and/or sewn up, if the physical object (the swab) cannot be visually detected by the camera but the last established location of the swab was in the zone in question.

Change data is also prepared which relates to changes in the status of the object. This change data can describe conditions in which the status of the at least one object changes. These conditions can for example be based on the object assuming the status of another object when the object touches or approaches another object and/or when a physical object is situated in and/or approaches a zone and/or when two zones touch and/or intersect and/or approach each other. Two zones approaching each other is in particular to be understood to mean that the outer boundaries of the zones, i.e. the peripheries, spatially approach each other at least two points (wherein one point respectively belongs to one of the two zones and/or lies on its periphery). The approach of two zones can however be defined as being that two points which lie in the interior of the two zones (i.e. one of the two points respectively belongs to one of the two zones but does not lie on its periphery) spatially approach each other. In the case of spherically or circularly defined zones, this can for example be their centre points, which spatially approach each other in a plane and/or in three-dimensional space. A physical object approaches a zone (which represents a non-physical object) when its location spatially approaches either the periphery of the zone or a point (defined in an analogous way to the approach of two zones) in the interior of the zone. This approach can also be described in two or three dimensions. A physical object has a spatial shape, i.e. can in particular be physically gripped. In this respect, a zone cannot be physically gripped (i.e. is not physical), since it is defined virtually. The approach of physical objects and zones as described above can be rendered in more concrete terms as follows: two zones can approach each other if for example a zone is defined around each of two instrument tables, relative to the location data of the instrument table. If, for example, these two instrument tables are pushed together (i.e. moved spatially nearer to each other), then the zones belonging to them are each moved together with the instrument tables, which results in an approach of the zones. If one of the two instrument tables is surrounded by a zone which has the “clean” status, and the other instrument table is surrounded by a zone which has the “unclean” status, then when the two zones overlap as a result of their approach, a change in the “clean” status to the “unclean” status is indicated. A physical object would for example be situated in the “unclean” zone if a “sterile” scalpel lay in this zone, i.e. if its location data describes coordinates which correspond to and/or are spatially encompassed by the coordinates from the location data of the “unclean” zone. A change in the status of the scalpel from “sterile” to “non-sterile” would then be indicated. Yet another status, “in danger of becoming non-sterile”, can also be used between the statuses of “sterile” and “non-sterile”. This additional status can then for example be assumed to obtain if two zones (one sterile zone and one non-sterile zone) touch. A corresponding warning signal can then be output which is based on the “in danger of becoming non-sterile” status. The zone can for example be recognized as “non-sterile” only once physical objects in the sterile zone and the non-sterile zone touch.

The change data can in particular also describe the status which the physical object and/or zone assumes or maintains when the condition for the change in status is fulfilled or not fulfilled. The object whose status is described is in particular situated in a medical space in which medical hygiene conditions are predetermined, for example in an operating theatre, a suture room, an examination room, a sickbay or in an ambulance. Thus, for example, a pair of tweezers which at first have the “sterile” status can assume the “non-sterile” status if they have touched a non-sterile environment and/or surface. This non-sterile surface can for example include the floor of an operating theatre, another non-sterile medical instrument, a non-sterile item of clothing belonging to the medical staff and/or a patient, a non-sterile liquid and/or a non-sterile storage surface.

The condition for the change in the status of the object can also be such that the object is changed to the “unusable” status once a particular period of time has elapsed. This is understood to mean that the object is no longer to be used and/or is no longer intended for use, in order for example to be able to keep to the working conditions (for example, requirements regarding sterility). This can for example apply to a drug or a liquid-impregnated compress, since these must not be exposed to the atmosphere for longer than a particular period of time, since otherwise they change their concentration of active agent, and therefore their usefulness, due to evaporation processes. The conditions for the change in status can also include a poll as to whether the object has location data which describes a location at which it must not be situated and/or is in fact supposed to be situated for particular reasons. On the basis of the conditions which are described in the change data, it is possible to poll whether a condition for the change in status obtains for the object. Thus, for example, the pair of tweezers need not necessarily change their “sterile” status and/or initial status if they come into contact with another object which also has the “sterile” status. A condition for the change in status would obtain in this case if the sterile pair of tweezers touched another object which had the “non-sterile” status. The pair of tweezers would then also have to assume the “non-sterile” status. Thus, the status of the object which is described by the object data is updated on the basis of the comparison between the change data and the object data. Thus, if the comparison between the object data and the change data reveals that the status of the object is to be changed, a change in the status to the status which follows from the change data is indicated; this change is made in accordance with the invention. Since statuses and ways of comparing them with each other can be defined in a variety of ways, statuses are designated purely as examples within the framework of the present text. Thus, a different designation and/or a change to a differently designated end status (i.e. the status after changing the initial status as a result of the comparison) is not excluded by the wording.

The object data can also be prepared in such a way that a movement of the at least one object can be reconstructed from it. The movement of a zone also includes a partial movement of a zone, i.e. for example a change in the shape of the zone. This can for example be achieved if the object data includes time information which is recorded in addition to the location data of the object, wherein the time information can be related to the location data of the object. From this data, a past movement of the object can then be retraced, recorded and/or reproduced. In particular, it thus becomes possible to define a series of location data (i.e. location information) of the object which enables the actual chronological sequence of the location data to be deduced. This can for example be reproduced using visual and/or audio-technical means; thus, for example, the spatial movement of the object can be visually displayed on a monitor. An audio signal, which is emitted by an audio reproduction system connected to a status updating system, can also describe the movement trajectory of the object. Extrapolating a future movement of the object on the basis of the object data is also part of the invention. By for example linearly extrapolating the movement, it is thus possible to ascertain, from the past movement trajectory, where the object will move next. This can serve the purpose of outputting a visual and/or audio-technical signal such as has just been described, which for example comprises a warning if the object is moving in a direction which would result in an undesirable change and/or update in the status of the object or of other objects and/or in a danger to people and/or material. The latter would for example apply to a sterile scalpel which slips from the surgeon's hand and falls towards the floor of an operating theatre, whereby it not only could become non-sterile but would also expose body parts of the medical staff and/or a patient (such as for example a foot) to the danger of injury.

In addition to the location data and status data of the at least one object, the object data can also include identification data with respect to the at least one object. This identification data can for example be captured via a camera, in an analogous way to the location data. In this case, shape and/or pattern recognition of the object or also the use of surfaces on the object exhibiting a particular electromagnetic reflection coefficient to be uniquely assigned to the object and/or its type (class) can in particular lead to an identification of the object. Identification data from RFID information and/or RFID tags attached to the object can also be evaluated by means of RFID sensors and in particular a data processing device, in order to identify the object. This identification data serves to uniquely identify the object and/or assign it to a type and/or class. If the object represents a medical instrument (i.e. is physical), it can thus be provided with a serial number which is for example arranged on the object in the form of writing and/or a barcode. It is however also possible to mould (for example, engrave or punch) a pattern and/or writing into the surface of the object, in order for this to then be detected (for example optically and/or mechanically). A comparison with a previously prepared database can then be used to assign this serial number or other marking to a particular dataset, whereupon the object can be identified and/or assigned to a type and/or class. If the object includes a zone, a marking in the form of the serial number described above (for example on an adhesive plate, or in the form of writing directly applied, for example engraved, on a solid surface of the zone) can be arranged in the region of this zone, wherein it is then possible to identify the zone in terms of other features. In the case of a zone, these other features can in particular be the compatibility of its surfaces with one or more cleaning agents, such that in the event of a change in status, for example from “clean” to “unclean”, a comparison can be made with identification comparative data, in order to calculate a required amount of cleaning agents in order to restore the original “clean” status. The identification data can however also be based on the geometry of the object, wherein in accordance with a method according to EP 100 900 336 A1, the outer shaping of the object leads to the identification of the object in a method based on optical technology. Methods from the field of shape recognition and/or pattern recognition can in particular be used to identify the object. The color of the object can also lead to the identification of the object, once it has been detected by a camera and a digital dataset based on the captured image has been evaluated. A surface characteristic of the object can also serve this purpose; for example, a particular electromagnetic reflection coefficient of the surface of the object can serve to identify and/or assign the object, for example with the aid of a spectroscopic method which evaluates light reflected by the surface in question. A marker device, which is attached to the object and known from navigation-assisted surgery methods and/or image-guided surgery (IGS) methods, can also be adduced in order to identify the object and/or zone, for example via a specific arrangement of the marker elements, their geometry, color and/or surface characteristic.

On the basis of the comparison between the object data and the change data, information on the update in the status of the object can be output. This information can for example be output in visual form, i.e. in the form of images and/or text, on a monitor. Outputting an audio signal which includes such information is also advantageous. A vibration signal can also for example indicate the information. The information can also be transmitted to a data processing device in the form of a signal and/or signal wave. The data processing device can then for example store the information and/or provide for reproducing it.

The object data can also be based on patient analysis data which relates to the location of the at least one object relative to a patient's body. The location data of the object in a patient's body can thus be ascertained with the aid of a patient analysis method, i.e. a method with the aid of which information on the geometry of a patient's body is obtained. This applies in particular when the object is situated at least partially within a patient's body; it is however also possible for the entire object to be situated within a patient's body. In this respect, the object data can be ascertained from and/or based on patient analysis data. Within the framework of surgical navigation methods, for example, patient analysis data is provided as a model of the patient's body. Together with the location data of the object, it is thus possible to determine an orientation of the object with respect to the patient's body. Such patient analysis data is usually prepared by imaging methods using x-ray radiation, in particular with the aid of computer tomographic and/or magnetic resonance tomographic data, and used as navigation data and/or image-guided surgery (IGS) data within the framework of surgical navigation methods. Ultrasound methods, conventional x-ray methods, positron emission tomography methods and single photon emission tomography methods may also for example be considered for capturing the patient analysis data.

The object data can also include target information and actual information on the geometry of the at least one object. The target information can for example include information which describes the geometry (shape) of the object, for example a medical instrument or implant, in a standard status, i.e. for example a status intended for use such as an undamaged status. The actual information can include information on how the actual current geometry (shape) of the object is represented. It is then for example possible to deduce any damage to (deformation of) a physical object and/or to deduce a geometric alteration to the zone from a comparison of the geometry information in the target information and the actual information. The object can then be assigned a particular status, i.e. for example “damaged” if there is damage.

The status and/or current status of the object can also be compared, for example in a database, with a nominal status of the object, wherein the nominal status can be based on information which includes a previously known location (detected for example before the beginning of the operation) and/or the usage status of the object. Once the current status and the nominal status of the object have been compared, it is possible to transmit information to a management unit, in order for example to ensure that similar objects and/or objects of the same type are reordered from a supplier or a cleaning order is issued for the zone. If, for example, the nominal status of the object is “unused”, but the current status is “used”, then it makes sense to transmit information to the management unit, indicating the need to reorder the (physical) object in question and/or to clean the zone.

The object data prepared in the method in accordance with the invention, i.e. the information on the location and status of the object, can be recorded. In addition to this information, chronological information corresponding to the location data and/or the status can be stored, in order to obtain a log, in particular a complete log, of the locations and statuses which the at least one object has passed through, in relation to their chronological sequence.

Depending on the comparison between the object data and the change data and/or depending on the result of this comparison, a control pulse can be emitted by a control device in order to trigger subsequent steps of the method. Using the method in accordance with the invention, it is for example possible to establish whether a surgeon's hand is operating an endoscope in such a way that it is clear that he wishes to use it on a patient. In order to provide better visibility for the application, the control device can transmit a control pulse to an illumination device and/or dimming device, in order to reduce the light intensity in the surgeon's working environment, such that he has a better view through the lens of the endoscope.

The method in accordance with the invention can be composed in the form of a program which can be stored in a permanent storage medium, from which it can be loaded onto a computer, such that it runs on a computer and causes the computer to perform the steps and/or actions mentioned above.

In one embodiment of the method in accordance with the invention, a camera array—in particular comprising at least two cameras which are spaced apart and are “looking” at the object—is used to optically (and spatially) detect an object and/or zone. The underlying principles are known for example from image-guided navigation (image-guided surgery). This can be performed for example using infrared radiation which is emitted by a transmitter and reflected by the object and detected by the camera array. Visible light, in particular also spatially present light (not for example originating from artificial light sources), can also be used in the same way. To this end, the object can be configured such that its surface in particular reflects infrared radiation and/or visible light in the desired way; to this end, reflective markings can for example be arranged on a physical object and/or in the zone (and/or at its outer boundary, i.e. on the periphery of the zone) and/or the reflection characteristic can be inherent in the surface material of the object. The distance from the camera or position with respect to the camera, for example the initial position at the beginning of the practical medical work, can be known in a coordinate system, for example a global coordinate system or relative to the camera array, or can be defined and/or ascertained in a calibration method, such that the spatial location data of the object and/or the zone can be captured by means of the camera array and/or alterations to this location can be detected and/or tracked by means of the camera array. The camera array can also identify a physical object by its shape using optical recognition methods (for example, pattern recognition methods) and determine its location. For recognition, the detected shape is in particular compared with stored shapes of the objects, such that for example a pair of scissors of a particular type are recognized by their shape.

If the object is detected using an individual camera, two spatial coordinates (and/or the spatial extent in two dimensions) of the object are directly ascertained; the third spatial coordinate (and/or the spatial extent in the third dimension) can for example be calculated from a change in the size of the object in the captured image. To this end, the detected shape of the object is compared with a previously known shape of the object in a known position relative to the camera; by means of elementary geometric considerations, the distance of the object relative to the camera array can then be ascertained from the deviating shape and/or size of the object in the captured image as compared to the previously known image.

If more than one camera is used in the camera array, the position of the at least two cameras of the camera array with respect to each other is preferably known or can in particular be determined in a calibration method. Such a method is described in EP 100 681 028 A1 (paragraphs [0007] to [0008]) and is incorporated into the disclosure of this invention. By using more than one camera, it is possible to capture the spatial location data of the object and/or the perspective from which the cameras record the object.

The camera or cameras of the camera array can in particular be designed such that they are designed for video recordings in the visible light range. It is thus possible to record objects and/or surfaces of objects which emit and/or reflect visible light. The camera array can also be configured such that it can take video recordings both in the visible light range and in the infrared range, in order in particular to be able to detect tracking markings which reflect and/or emit infrared radiation (i.e. passive and/or active marker devices), such as markers or reference stars. The camera array can also be arranged such that it is stationary or such that it can be moved. If the camera array is designed such that it can be moved, the camera position should be recalibrated after each change in the location of the camera array relative to the previously calibrated position.

A dataset which relates to the change data is also stored in a data storage device which in particular includes a permanent data memory. This dataset includes coordinates which describe conditions in which the status of the at least one object changes. Fulfilling these conditions can in particular be based on whether at least two objects come into contact, in particular physical contact, with each other. Fulfilling these conditions can also be based on whether an object is situated in a zone and/or whether two zones intersect and/or touch each other. Two mutually intersecting zones can intersect in such a way that they have a common straight-line intersection and/or straight-line intersection of limited extent if at least one of the two zones is defined as an area or plane, i.e. two-dimensionally (i.e. having spatial coordinates in which at least one coordinate is the same for all the points of the zone). If both zones are defined as spatial zones, i.e. three-dimensionally, their intersection in turn results in a spatial zone, i.e. a zone which has limits which can be described using coordinates in three dimensions, wherein none of the coordinates need be fixed. In accordance with the invention, an object is situated in a zone if the zone is two-dimensionally defined and the location of the object lies within the area and/or plane thus defined. In the case of a three-dimensionally defined zone, an object lies within the zone if the location of the object is situated within and/or on the limit of the spatial portion thus defined. If the limits of a number of two-dimensionally and/or three-dimensionally defined zones lie on top of one other, and an object is situated on such a common limit, then the changing condition can expediently prescribe that a particular status, which belongs to at least one of the number of zones, can be assigned to the object. If, for example, an object which has the “sterile” status is situated on the common limit of a zone which has the “sterile” status and a zone which has the “non-sterile” status, the changing condition can stipulate—as a safety measure—that the object is assigned the “non-sterile” status. The coordinates can be described as multi-dimensional Cartesian coordinates, polar coordinates or spherical coordinates and/or in the form of vectors (i.e. described by a direction value and a distance value, for example relative to the camera array). A data processing device, which in particular includes a permanent data memory and a processor, can be connected to the camera array. The data processing device makes the comparison between the object data and the change data and can output results and/or information on the basis of this. These results can in particular indicate that the status of the object is changed or maintained. The data processing device can also record location data and/or status data and/or reproduce recorded location data and/or status data. Previously known data on objects, such as their status data and/or location data, can also be stored in the data memory and read by the data processing device for the comparison. Such previously known and/or predetermined location data can in particular include information on locations at which a physical object is not to be situated. By capturing the location data and comparing it with the predetermined location data, it is in particular possible to ascertain whether all the physical objects (for example, medical instruments) are situated at an assigned location after an operation has been concluded. It is thus for example possible to prevent instruments from unintentionally remaining in the patient's body when an operation is concluded.

### BRIEF DESCRIPTION OF THE DRAWINGS

FIG. 1 shows an instrument table comprising physical objects and differently defined zones on the table top.

FIG. 2 shows a camera array such as is usually used in surgical navigation methods.

FIG. 3 shows an instrument table comprising physical objects and differently defined zones on the table top and on an individual camera array.

FIG. 4 shows a possible environment in which a medically usable, physical object is used within the framework of the method in accordance with the invention.

FIG. 5 shows a workflow for the method in accordance with the invention for changing the status of objects.

### DETAILED DESCRIPTION

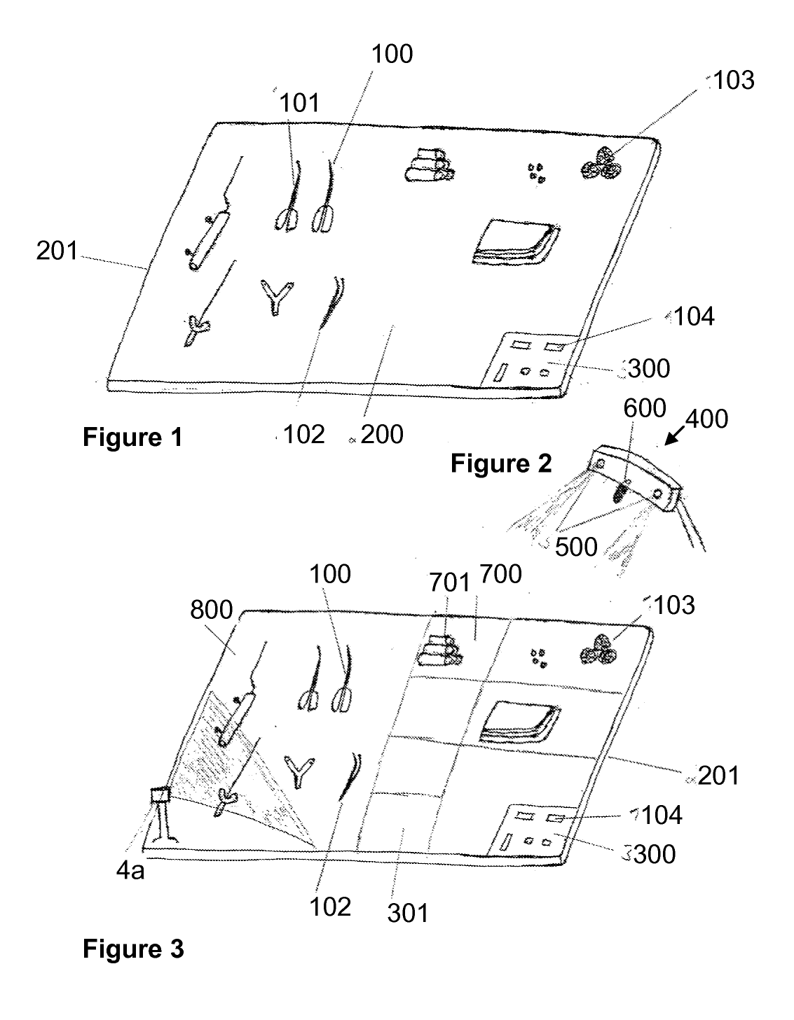

FIG. 1 shows an instrument table 201 on which different medical objects 100, 103 and 104 are situated. The objects 100 and 103 are situated in a zone of the table top of the instrument table 201, i.e. in a zone of a surface of the instrument table 201. This zone is a clean zone 200, i.e. a zone which may be regarded as being in particular clean. The object 104 is situated in a zone of the table top, i.e. in a zone of a surface of the instrument table 201, which is regarded as an unclean zone 300, i.e. as a zone which may be regarded as being in particular unclean. Objects 100, 103 and the object 104 can for example be stored in the unclean zone 300 when they have and/or have assumed a non-sterile status and/or a non-use status, i.e. a status in which they are not intended for use.

FIG. 2 shows a camera array 400 such as it usually used in image-guided surgery (IGS) methods and/or in surgical navigation methods. The camera array 400 includes a transmitter 600 which can emit electromagnetic radiation. This electromagnetic radiation is preferably infrared radiation and/or visible light. The camera array 400 also includes at least one—in the scenario shown, two—receivers and/or cameras 500 which are capable of receiving electromagnetic radiation, preferably infrared radiation and/or visible light. It is also possible for a number of transmitters 600 to be arranged in the camera array 400, for example one transmitter 600 for each camera 500, wherein a transmitter 600 can be arranged annularly around a lens aperture and/or receiver aperture of the camera 500. The infrared radiation and/or visible light which is emitted by the transmitter 600 can for example be reflected by a surface of the instrument table 201 and/or of an object 100, 103, 104. The reflected electromagnetic radiation can then be recorded in cameras 500, whereupon a corresponding signal can be transferred to a data processing device and/or computer 1900. From this signal, the data processing device 1900 can for example calculate the location data of the surface and/or object from which the electromagnetic radiation was reflected, i.e. for example the location data of the operating table 201, the objects 100, 103, 104 and/or the clean zone 200 and/or the unclean zone 300. For this purpose, the surfaces of the reflective object can be embodied differently, in order to reflect electromagnetic radiation—in particular in the infrared and/or visible range—differently. This enables the object to which the reflecting surface belongs to be identified by the data processing device 1900. To this end, the data processing device 1900 compares the characteristics of the signal emitted by the camera array 400 in accordance with the recorded electromagnetic radiation with previously stored characteristics of the electromagnetic radiation reflected by the surfaces of the objects 210, 100, 103, 104, in particular in the infrared and/or visible range. This enables the reflecting object to be identified. This identification can be made individually according to each object (for example, a distinction can be made between two pairs of scissors 100 and 101 of the same type) or according to the type and/or class of the object (a distinction can be made between a pair of scissors 100 and a swab 103).

FIG. 3 shows an individual camera array 401 which is fastened to an instrument table 201 (i.e. is in physical contact with an instrument table 201). Zones of the instrument table 201 are defined on its surface and for example include the clean zones 700, 800. An unclean zone 300 is also defined. One zone 301 is for example not assigned a status. The user 1000 can in particular assign this zone 301 a status by making a corresponding input on an input device 1904 of a data processing device 1900. The individual camera array 401 can be designed and/or arranged to irradiate only one of the zones 300, 700, 800 with electromagnetic radiation by means of a transmitter 600 which it includes, and to receive electromagnetic radiation reflected by the zone in question. Such a zone 300, 700, 800 can in particular include the surface of a storage option. The individual camera array 401 corresponds in its design to the camera array 400 from FIG. 2. The individual camera array 401 thus detects and/or tracks, within the zone 800 which it is viewing, only objects which—like the object 100—are situated in a two-dimensional projection of the zone 800 onto the field of view of the camera array 401. If the camera array 401 is designed to monitor only the clean zone 700, then it only detects and/or tracks objects 701 which are situated in a two-dimensional projection of the clean zone 700 onto the field of view of the camera array 401. Similarly, if the camera array 401 is designed to monitor only the unclean zone 300, it only detects objects 104 which are situated in a two-dimensional projection of the unclean zone 300 onto the field of view of the camera array 401.

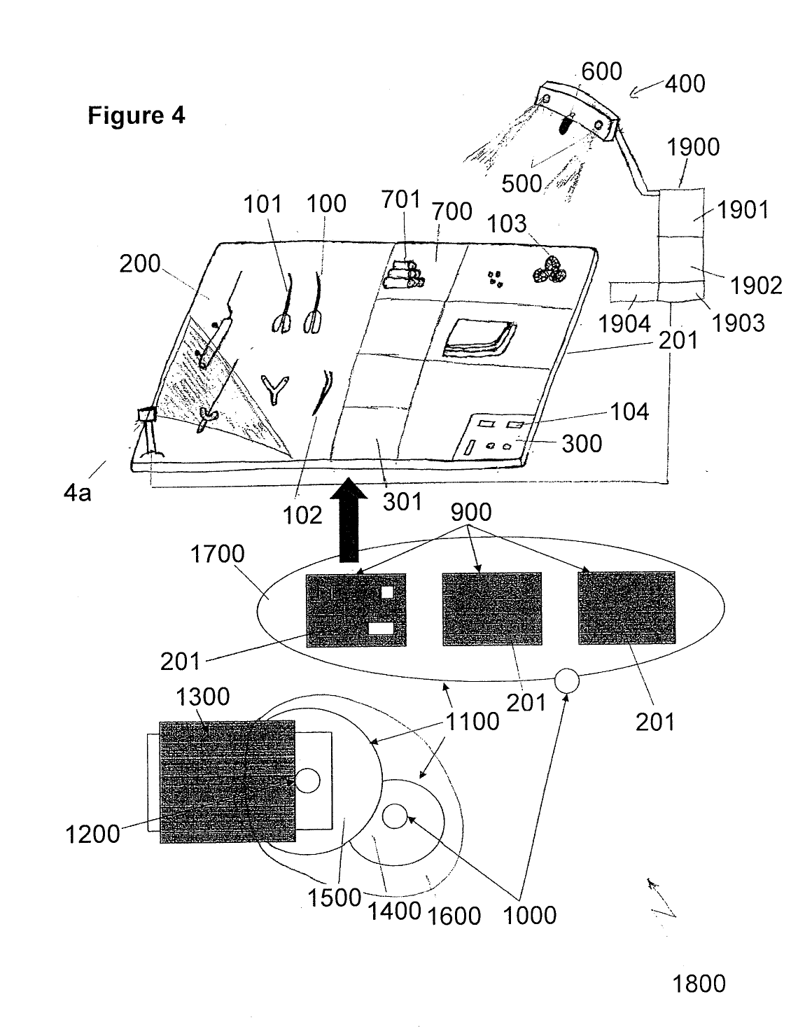

FIG. 4 schematically shows the design of a working environment 1800 in which the method in accordance with the invention is typically used. This schematic design includes medical staff 1000, zones 1100, the head or a body part 1200 of a patient, covering material 1300 and instrument tables 900. Each of the instrument tables 900 shown is identical in its essential design to the instrument table 201 already presented in FIG. 3. Zones 1100 can be defined around each object present in the working environment 1800 and are assigned a particular status (such as for example “sterile” or “non-sterile” and/or “clean” or “unclean” and/or “usable” or “unusable” and/or “currently in use” or “used” or “unused” and/or “at rest” or “in motion”). This status of the zones 1100 can in particular be identical to the status of the object relative to which it is defined. This means that all the spatial coordinates which are situated in the zones 1100 are assigned the respective status of the zone 1100. The zones 1100 can extend two-dimensionally and/or three-dimensionally. By way of example, FIG. 4 shows such zones around the body part 1200 of a patient (zone 1500), the location of medical staff 1000 (zone 1400) and around a multitude 900 of instrument tables 201 (zone 1700). Zones 200, 300, 700 can in turn be defined on each of the instrument tables 201, wherein in the embodiment of FIG. 4, each of the zones 200, 300, 700 and/or 1100 can be optically detected by a camera array 401 designed especially for monitoring it alone. It is however also possible for a plurality of zones 200, 300, 700, 1100 and/or all the zones 200, 300, 700, 1100 to be optically detected by one camera array 400. Individual zones 1100 can also be combined to form a common zone. This can be achieved, as in the case of the zones 1400 and 1500, by defining a zone 1600 which encompasses the zones 1400 and 1500. It is also conceivable to define a new zone 1600 on the basis of an intersection between two three-dimensional zones 1400 and 1500. In the case of two-dimensional zones 1400 and 1500, however, the zone 1600 can also represent the intersection between the two-dimensional zones 1400 and 1500, wherein the intersection in this case can take the form of a straight-line intersection (if the zones 1400 and 1500 are defined as planes) or a straight-line intersection of limited extent (if the zones 1400 and 1500 are defined as areas). It can also be the case that two zones consisting of the plurality 1100 of zones 1500, 1600, 1700 are shifted—by a shift in an object around which they can be defined—such that they enter another zone. This would for example be the case if the medical staff 1000, around whom the zone 1400 is for example defined at a fixed radius, moves near enough to the body part 1200 of the patient, around which the zone 1500 is defined at a fixed radius, that the zones 1400 and 1500 would intersect each other. If this should be avoided, since the zone 1500 is for example defined as being clean and the zone 1400 is for example defined as being unclean, it is for example possible in accordance with the invention to define only the part of the zone 1400 which intersects with the zone 1500 as corresponding to the zone 1500.

The possible reference signs for an object 100, 101, 102, 103, 104, 701, 1000, 1200, 1300, 200, 300, 301, 700, 1400, 1500, 1600, 1700 are shortened to “X” in the following, i.e. “the object X” is written instead of “the object 100, 101, 102, 103, 104, 701, 1000, 1200, 1300, 200, 300, 301, 700, 1400, 1500, 1600, 1700”.

The possible reference signs for a (physical) object 100, 101, 102, 103, 104, 201, 701, 1200, 1300 are shortened to “Y” in the following, i.e. “the object Y” is written instead of “the object 100, 101, 102, 103, 104, 201, 701, 1200, 1300”.

The possible reference signs for a zone 200, 300, 700, 1400, 1500, 1600, 1700 are shortened to “Z” in the following, i.e. “the zone Z” is written instead of “the zone 200, 300, 700, 1400, 1500, 1600, 1700”.

The location data of an object X can be understood to mean a description of the location of the object X, for example with the aid of multi-dimensional coordinates (for example, Cartesian coordinates, polar coordinates and/or spherical coordinates) and/or vectors (which indicate the location of the object X by their direction with respect to a reference location, for example the location of the camera array 400, 401, and a distance from said reference location). The location data of the object X can be specified in a global coordinate system or also relative to another object X which can differ from the first object X. An orientation of the object X, in particular relative to another object X, can thus be specified (for example, in the form of vectors).

In accordance with the invention, an initial number of objects Y can be ascertained by a data processing device 1901, and for example with the aid of the camera array 400, at a start time of an operation. An end number of objects Y can also be ascertained at an end time of an operation. It is also possible to ascertain an intermediate number of objects Y at a point in time which lies between the start time and the end time of an operation. The intermediate number and/or the end number of objects Y can then be compared with the initial number of objects Y, wherein a notification can be output to a user on the basis of the result of this comparison. The comparison between the intermediate number and/or the end number and the initial number can be made by a processor 1901, wherein the values to be compared can for example be stored in a data memory 1902. The start time and/or end time of the operation and/or the initial number and/or intermediate number and/or end number of objects Y can be automatically ascertained by the data processing device or also input via a manual input by the user by means of an input device 1904. The notifying information, which is based on the comparison of the numbers and can for example include information on an undesirable deviation between the numbers, can for example be indicated in visual form and/or as an audio signal in an indicating device 1903 which can for example be a monitor and/or can include a loudspeaker. The initial number can be valid for the entire space (for example, the operating theatre) and can in particular correspond to the initial total number of objects. It can however also be predetermined for individual zones. The intermediate number or end number is correspondingly determined for individual zones (for example, the operating environment). In the case of the operating environment zone, the initial number is in particular zero.

The status of the object X relates to and in particular describes for example its applicability, i.e. it includes information for example with respect to the sterility or non-sterility of the object Y, the intactness of or damage to the object X, the cleanness or uncleanness of the object X, the use or non-use of the object X, for example whether it is currently being used (i.e. for example in the case of a physical object Y, whether it is in direct and/or indirect contact, for example physical contact with a user 1000 and/or whether the user 1000 is situated in the zone Z), has been used or has not yet been used and/or can be used or cannot be used. In this context, “use” means at least that the object X is used in a way in which the use is intended. In this way, the position of the object X in the workflow can be described, and the object X which is to be used next in the workflow can for example be ascertained. On the basis of this ascertainment, it is possible to indicate to the user 1000 which object X is to be used next. Change data is also prepared which relates to changes in the status of the object X. This change data can describe conditions in which the status of the at least one object X changes. These conditions can for example be based on the object Y assuming the status of another object Y when it touches or approaches the other object Y and/or when the object Y is situated in and/or approaches a zone Z and/or when two zones Z touch and/or intersect and/or approach each other. Two zones Z approaching each other is in particular to be understood to mean that the outer boundaries of the zones Z, i.e. the peripheries, spatially approach each other at least two points (wherein one point respectively belongs to one of the two zones and/or lies on its periphery). The approach of two zones Z can also be defined as being that two points which lie in the interior of the two zones Z (i.e. one of the two points respectively belongs to one of the two zones Z but does not lie on its periphery) spatially approach each other. In the case of spherically or circularly defined zones Z, this can for example be their centre points, which spatially approach each other in the plane and/or in three-dimensional space. An object Y approaches a zone Z when its location spatially approaches either the periphery of the zone Z or a point (defined in an analogous way to the approach of two zones Z) in the interior of the zone Z. This approach can also be described in two or three dimensions. The change data can in particular also describe the status which the object X assumes or maintains when the condition for the change in status is fulfilled or not fulfilled. The object X whose status is described is in particular situated in a medical space 1800 in which medical hygiene conditions are predetermined, for example in an operating theatre, a suture room, an examination room, a sickbay or in an ambulance. Thus, for example, a pair of tweezers 102 which at first have the “sterile” status can assume the “non-sterile” status if they have touched a non-sterile environment and/or surface. This non-sterile surface can for example include the floor of an operating theatre and/or another non-sterile medical instrument Y and/or a non-sterile item of clothing belonging to the medical staff 1000 and/or a patient and/or a non-sterile liquid and/or a non-sterile storage surface 300 and/or a non-sterile covering 1300 and/or a non-sterile body part 1200 of a patient and/or staff member 1000. The condition for the change in the status of the object X can also be such that the object X is changed to the “unusable” status once a particular period of time has elapsed. This is understood to mean that the object X is no longer to be used and/or is no longer intended for use, in order for example to be able to keep to the working conditions (for example, requirements regarding sterility). This can for example apply to a drug or a liquid-impregnated compress, since these must not be exposed to the atmosphere for longer than a particular period of time, since otherwise they change their concentration of active agent, and therefore their usefulness, due to evaporation processes. The conditions for the change in status can also include a poll as to whether the object Y is situated at a location and/or whether the zone Z has coordinates at which it must not be situated and/or is in fact supposed to be situated for particular reasons. On the basis of the conditions which are described in the change data, it is possible to poll whether a condition for the change in status obtains for the object X. Thus, for example, the pair of tweezers 102 need not necessarily change their “sterile” status and/or initial status if they come into contact with another object X which also has the “sterile” status. A condition for the change in status would obtain in this case if the sterile pair of tweezers 102 touched another object X which had the “non-sterile” status. The pair of tweezers 102 would then also have to assume the “non-sterile” status. Thus, the status of the object X which is described by the object data is updated on the basis of the comparison between the change data and the object data. Thus, if the comparison between the object data and the change data reveals that the status of the object X is to be changed, a change and/or update in the status to the status which follows from the change data is indicated; this change and/or update is made in accordance with the invention. In this context, an object Y is also to be understood to mean a zone Z. If, for example, an object which has the “unclean” status is situated in a clean zone 200, 700, 1400, 1500, 1600, 1700 and if this is established within the framework of the method in accordance with the invention, then the status of the clean zone 200, 700, 1400, 1500, 1600, 1700 can be changed and/or updated to “unclean” in accordance with the invention, i.e. a clean zone 200, 700, 1400, 1500, 1600, 1700 can be made into an unclean zone 200, 700, 1400, 1500, 1600, 1700. The objects Y can also then be assigned the “unclean” status and/or their previous status can be changed and/or updated to the “unclean” status if they were situated in the clean zone 200, 700, 1400, 1500, 1600, 1700 at the time its status was changed and/or updated from “clean” to “unclean”. The “clean” status of a clean zone 200, 700, 1400, 1500, 1600, 1700 can also be changed to “unclean” if it intersects with an unclean zone 300, 1400, 1500, 1600, 1700. In this case, the “clean” status of the clean zone 200, 700, 1400, 1500, 1600, 1700 is changed and/or updated to “unclean”. The status of a clean zone 200, 700, 1400, 1500, 1600, 1700 which is assigned the “clean” status can also be changed to “unclean” if an object Y is situated in it which has the “unclean” status. It is however also possible for only the status of the part of the clean zone 200, 700, 1400, 1500, 1600, 1700 which is situated in the intersection region between the clean zone 200, 700, 1400, 1500, 1600, 1700 and the unclean zone 300, 1400, 1500, 1600, 1700 to be changed and/or updated to “unclean”. The part of the clean zone 200, 700, 1400, 1500, 1600, 1700 which is situated in the intersection region between the clean zone 200, 700, 1400, 1500, 1600, 1700 and the unclean zone 300, 1400, 1500, 1600, 1700 can then for example be defined as a new unclean zone 300, 1400, 1500, 1600, 1700 or can be assigned to an existing unclean zone 300, 1400, 1500, 1600, 1700, i.e. it is defined as a part of and/or as belonging to the existing unclean zone 300, 1400, 1500, 1600, 1700.

The object data can also be prepared in such a way that a movement of the at least one object Y can be reconstructed from it. This can for example be achieved if the object data includes time information which is recorded in addition to the location data of the object Y. In this context, an object Y is also to be understood to mean a zone Z. The movement of a zone Z also includes a partial movement of a zone Z, i.e. for example a change in the shape of the zone Z, wherein the time information can be related to the location data of the object X. From this data, a past movement of the object X can then be retraced, recorded and/or reproduced. In particular, it thus becomes possible to define a series of location data and/or location information of the object X which enables the actual chronological sequence of the location data to be deduced. This can for example be reproduced using visual and/or audio-technical means; thus, for example, the spatial movement of the object X can be visually displayed on a monitor. An audio signal, which is emitted by an audio reproduction system connected to a status updating system, can also describe the movement trajectory of the object X. Extrapolating a future movement of the object X on the basis of the object data is also part of the invention. By for example linearly extrapolating the movement, it is thus possible to ascertain, from the past movement trajectory, where the object X will move next. This can serve the purpose of outputting a visual and/or audio-technical signal such as has just been described, which for example comprises a warning if the object X is moving in a direction which would result in an undesirable change and/or update in the status of the object X or of other objects X and/or in a danger to people 1000 and/or material 1300. The latter would for example apply to a sterile scalpel which slips from the staff's 1000 hand and falls towards the floor of an operating theatre, whereby it not only could become non-sterile (i.e. its status would for example be changed and/or updated from “sterile” to “non-sterile”) but would also expose body parts of the medical staff 1000 and/or a patient (such as for example a head 1200) to the danger of injury.

In addition to the location data and status data of the object X, the object data can also include identification data with respect to the at least one object X. These identification data serves to uniquely identify the object X and/or assign it to a type and/or class. If the object represents a medical instrument 100, 101, 701, 103, 104, 1300 (i.e. also a physical object Y), it can thus be provided with a serial number which is for example arranged on the object Y in the form of writing and/or a barcode. It is however also possible to mould (for example, engrave or punch) a pattern and/or writing into the surface of the object Y, in order for this to then be detected (for example optically and/or mechanically). A comparison with a previously prepared database which includes identification comparative data can then be used to assign this serial number or other marking to a particular identification comparative dataset, whereupon the object Y can be identified and/or assigned to a type and/or class. If the object X includes a zone Z (i.e. a non-physical object), a marking in the form of the serial number described above (for example on an adhesive plate, or in the form of writing directly applied, for example engraved, on a solid surface of the zone) can be arranged in the region of this zone Z, wherein it is then possible to identify the zone Z in terms of other features. In the case of a zone Z, these other features can in particular be the compatibility of its surfaces with one or more cleaning agents, such that in the event of a change in status, for example from “clean” to “unclean”, a comparison can be made with the identification comparative data, in order to calculate a required amount of cleaning agents in order to restore the original “clean” status. The identification data can however also be based on the geometry of the object X, wherein in accordance with a method according to EP 100 900 336 A1, the outer shaping of the object X leads to the identification of the object X in a method based on optical technology. Methods from the field of shape recognition and/or pattern recognition can in particular be used to identify the object X. The color of the object Y can also lead to the identification of the object Y, once it has been detected by a camera and a digital dataset based on the captured image has been evaluated. A surface characteristic of the object 100, 101, 102, 103, 104, 701, 1300 can also serve this purpose; for example, a particular electromagnetic reflection coefficient of the surface of the object Y can serve to identify and/or assign the object Y, for example with the aid of a spectroscopic method which evaluates light reflected by the surface in question. A marker device, which is attached to the object Y and/or arranged in the zone Z and is known from navigation-assisted surgery methods and/or image-guided surgery (IGS) methods, can also be adduced in order to identify the object X, for example via a specific arrangement of the marker elements, their geometry, color and/or surface characteristic.

On the basis of the comparison between the object data and the change data, information on the update in the status of the object X can be output. This information can for example be output in visual form, i.e. in the form of images and/or text, on a monitor. Outputting an audio signal which includes such information is also advantageous. A vibration signal which is generated by a vibration device can also for example indicate the information. Such a vibration device can be in contact, for example physical contact, with at least one staff member 1000, such that a vibration of the vibration device can be perceived by said staff member 1000. The information can also be transmitted to a data processing device 1900 in the form of a signal and/or signal wave. The data processing device 1900 can then for example store the information and/or provide for reproducing it.

The object data can also be based on patient analysis data which relates to the location of the at least one object X relative to a patient's body. The location data of the object X in a patient's body can thus be ascertained with the aid of a patient analysis method, i.e. a method with the aid of which information on the geometry of a patient's body is obtained. This applies in particular when the object X is situated at least partially within a patient's body; it is however also possible for the entire object X to be situated within a patient's body. In this respect, the object data can be ascertained from and/or based on patient analysis data. Within the framework of surgical navigation methods, for example, patient analysis data is provided as a model of the patient's body and/or a part 1200 of the patient's body. Together with the location data of the object X, it is thus possible to determine an orientation of the object X with respect to the patient's body. Such patient analysis data is usually prepared by imaging methods using x-ray radiation, in particular with the aid of computer tomographic and/or magnetic resonance tomographic data, and used as navigation data and/or image-guided surgery (IGS) data within the framework of surgical navigation methods. Ultrasound methods, conventional x-ray methods, positron emission tomography methods and single photon emission tomography methods may also for example be considered for capturing the patient analysis data.

The object data can also include target information and actual information on the geometry of the at least one object X. The target information can for example include information which describes the geometry of the object Y, for example a medical instrument Y (i.e. a physical object) or implant, in a standard status, i.e. for example a status intended for use such as an undamaged status. The actual information can include information on how the actual current geometry of the object X is represented. It is then for example possible to deduce any damage to the object Y and/or to deduce a geometric alteration to the zone Z from a comparison of the geometry information in the target information and the actual information. The object X can then be assigned a particular status, i.e. for example “damaged” if there is damage.

The status and/or current status of the object X can also be compared, for example in a database, with a nominal status of the object X, wherein the nominal status can be based on information which includes a previously known location (detected for example before the beginning of the operation) and/or the usage status of the object X. Once the current status and the nominal status of the object Y have been compared, it is possible to transmit information to a management unit, in order for example to ensure that similar objects Y and/or objects Y of the same type are reordered from a supplier. If, for example, the nominal status of the object is “unused”, but the current status is “used”, then information is preferably transmitted to the management unit, indicating the need to reorder the object Y in question.

The object data prepared in the method in accordance with the invention, i.e. the information on the location and status of the object X, can be recorded. In addition to this information, chronological information corresponding to the location data and/or the status can be stored, in order to obtain a log, in particular a complete log, of the location data and statuses which the at least one object X has passed through, in relation to their chronological sequence. The determined periods of time which have been determined for the individual statuses and/or the times of the changes in status can in particular be used in order to compare them with the corresponding planned values. Minimum or maximum periods of use can for example be planned for particular objects and compared with the current periods of use. If, for example, a swab exceeds the planned period of use, then notifying information can be output on the basis of this, since the swab may have been forgotten (for example, in the patient's body). This notifying information (or also other notifying information) can in particular contain information on the location in which (i.e. at which) the object was last detected by the detection device (for example, a camera and navigation system).

Depending on the comparison between the object data and the change data and/or depending on the result of this comparison, a control pulse can be emitted by a control device (a processor 1901) in order to trigger subsequent steps of the method. Using the method in accordance with the invention, it is for example possible to establish whether a surgeon's 1000 hand is touching an endoscope or operating it in such a way that it is clear that he wishes to use it on a patient. In order to provide better visibility for the application, the control device (a processor 1901) could transmit a control pulse to apparatuses of the treatment room, in particular to an illumination device and/or dimming device, in order to reduce the light intensity in the surgeon's 1000 working environment 1800, such that he has a better view through the lens of the endoscope.

The method in accordance with the invention can be composed in the form of a program which can be stored in a permanent storage medium, from which it can be loaded onto an electronic data processing device 1900 and/or computer, such that it runs on a computer and causes the computer to perform the steps and/or actions mentioned above.

In the method in accordance with the invention, a camera array 400, 401—in particular comprising at least two cameras 500—is used to optically detect an object X. This can be performed for example using infrared radiation which is emitted by a transmitter 600 and reflected by the object X and detected by the camera array 400, 401. Visible light, in particular also spatially present light (not for example originating from artificial light sources), can also be used in the same way. To this end, the object X can be configured such that its surface in particular reflects infrared radiation and/or visible light in such a way that the reflected electromagnetic radiation and/or waves can be detected by the camera or cameras 500; to this end, reflective markings can for example be arranged on the object Y and/or in the zone Z (and/or at its outer boundary, i.e. on the periphery of the zone Z) and/or the reflection characteristic can be inherent in the surface material of the object. The distance or the position, for example the initial position at the beginning of the practical medical work, can be known in a coordinate system, for example a global coordinate system or relative to the camera array 400, 401, or can be defined and/or ascertained in a calibration method, such that the spatial location data of the object X can be captured by means of the camera array 400, 401 and/or alterations to this location can be detected and/or tracked by means of the camera array 400, 401.

If the object X is detected using an individual camera 500, two spatial coordinates of the object X are directly ascertained; the third spatial coordinate can for example be calculated from a change in the size of the object X in the captured image. To this end, the detected shape of the object X is compared with a previously known shape of the object X in a known position relative to the camera 500; by means of elementary geometric considerations, the distance of the object X relative to the camera array 400, 401 can then be ascertained from the deviating shape and/or size of the object X in the captured image as compared to the previously known image.

If more than one camera 500 is used in the camera array 400, 401, the position of the at least two cameras 500 of the camera array 400, 401 with respect to each other is preferably known or can in particular be determined in a calibration method. Such a method is described in EP 100 681 028 A1 (paragraphs [0007] to [0008]) and is incorporated into the disclosure of this invention. By using more than one camera 500, it is possible to capture the spatial location data of the object X and/or the perspective from which the cameras 500 record the object X.

The camera 500 or cameras 500 of the camera array 400, 401 can in particular be designed such that they are designed for video recordings in the visible light range. It is thus possible to record objects Y and/or surfaces of objects Y and/or zones Z which emit and/or reflect visible light. The camera array 400, 401 can also be configured such that it can take video recordings both in the visible light range and in the infrared range, in order in particular to be able to detect tracking markings which reflect and/or emit infrared radiation (i.e. passive and/or active marker devices), such as markers or reference stars. The camera array 400, 401 can also be arranged such that it is stationary or such that it can be moved. If the camera array 400, 401 is designed such that it can be moved, the camera position 400, 401 should be recalibrated after each change in the location of the camera array 400, 401 relative to the previously calibrated position.

A dataset which relates to the change data is also stored in a data storage device which in particular includes a permanent data memory and/or permanent storage medium 1902. This dataset includes coordinates which describe conditions in which the status of the at least one object Y and/or the at least one zone Z changes. Fulfilling these conditions can in particular be based on whether at least two objects Y come into contact, in particular physical contact, with each other. Fulfilling these conditions can also be based on whether an object Y is situated in a zone Z and/or whether two zones Z intersect each other. Two mutually intersecting zones Z can intersect in such a way that they have a common straight-line intersection and/or straight-line intersection of limited extent if at least one of the two zones is defined as an area or plane, i.e. two-dimensionally (i.e. having spatial coordinates in which at least one coordinate is the same for all the points of the zone). If both zones Z are defined as spatial zones, i.e. three-dimensionally, their intersection in turn results in a spatial zone, i.e. a zone Z which has limits which can be described using coordinates in three dimensions, wherein none of the coordinates need be fixed. In accordance with the invention, a physical object Y is situated in a zone Z if the zone Z is two-dimensionally defined and the location of the object Y lies within the area and/or plane Z thus defined. In the case of a three-dimensionally defined zone Z, an object Y lies within the zone Z if the location of the object Y is situated within and/or on the limit of the spatial portion Z thus defined. If the limits of a number of two-dimensionally and/or three-dimensionally defined zones Z lie on top of one other, and a physical object Y is situated on such a common limit, then the changing condition can expediently prescribe that a particular status, which belongs to at least one of the number of zones Z, can be assigned to the object Y. If, for example, an object Y which has the “sterile” status is situated on the common limit of a zone Z which has the “sterile” status and a zone Z which has the “non-sterile” status, the changing condition can stipulate—as a safety measure—that the object Y is assigned the “non-sterile” status. The coordinates can be described as multi-dimensional Cartesian coordinates, polar coordinates or spherical coordinates and/or in the form of vectors (i.e. described by a direction value and a distance value, for example relative to the camera array 400, 401). A data processing device 1900, which in particular includes a permanent data memory 1902 and a processor 1901, can be connected to the camera array 400, 401. The data processing device 1900 makes the comparison between the object data and the change data and can output results and/or information on the basis of this. The data processing device 1900 can also record location data and/or status data and/or reproduce recorded location data and/or status data. Previously known data on objects X, such as their status data and/or location data, can also be stored in the data memory 1902 and read by the data processing device 1900 for the comparison. Such previously known and/or predetermined location data can in particular include information on locations at which a physical object Y is not to be situated. By capturing the location data and comparing it with the predetermined location data, it is in particular possible to ascertain whether all the physical objects (for example, medical instruments) Y are situated at an assigned location after an operation has been concluded. It is thus for example possible to prevent instruments from unintentionally remaining in the patient's body when an operation is concluded.

The planned sequence of statuses for different objects can in particular include an initial location having an initial status and an end location having an end status. This can for example be defined for each object, in particular for each instrument. Thus, for example, the initial location of a pair of scissors can be on a table, as shown in FIG. 1. The initial status is “not yet used”. The planned end status is “already used” and the end location can for example be on another table, on which instruments which are no longer sterile are stored. Correspondingly, in the case of single-use objects for example, the location of waste containers can be defined as the end location for these single-use objects. It is in particular possible to determine that the object has reached the end location and the planned end status when it enters a zone around the waste container. Optical detectors and cameras can also in particular be attached to the waste containers for this purpose. The detection device which includes these cameras can then recognize the used objects by their shape and, as applicable, by their color and can recognize their location as being situated within the “waste zone”.

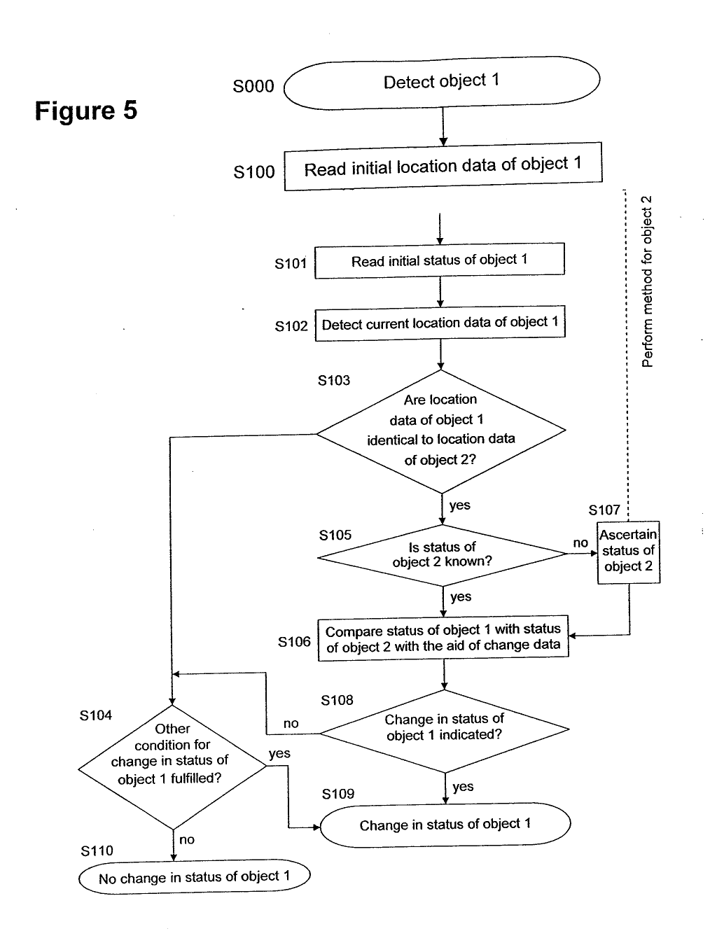

FIG. 5 summarizes the method described above in a workflow, which ascertains whether a condition for the change in status is fulfilled for an object. Step S000 comprises optically detecting an object X using the camera device 400, 401. In Step S100, the processor 1901 reads the previously known location data of an object X (defined for example at the beginning of the operation) from the data memory 1902. In Step S101, the processor 1901 reads the previously known initial status of the object X. In Step S102, the current location data of the object X is then read by the processor 1901 by means of the camera device 400, 401 and advantageously stored in the data memory for subsequent use.

In Step S103, a poll is taken as to whether the location information in the location data of the object X is identical to and/or encompassed by the location information in the location data of another object X. The location data of the other object X is either captured in this Step S103 by means of the camera device 400, 401 or is previously known and is read from the data memory 1902 by the processor 1901.

If the result of Step S103 is that the location data of the object X is identical to and/or encompassed by the location data of another object X, a poll is taken in Step S105 as to whether the status of the other object X is known. If this status is not known (for example from a database stored in the data memory 1902), it is ascertained in Step S107. This can for example be achieved by entering Step S100 in order to ascertain the status of the other object X.

If the status of the other object X is then ascertained and/or was ascertained to be known in Step S105, then the status of the object X is ascertained in Step S106 with the status of the other object on the basis of available changing conditions from the change data, i.e. a check is made as to whether a corresponding and/or opposing status of the other object X is available for a status of the object X and whether a changing condition is available in the change data for this combination of statuses. Whether a condition for the change in status of the object X is fulfilled is ascertained in Step S108 with the aid of a poll from the database in the data memory 1902, in which the change data is stored.

If said changing condition is fulfilled, the status of the object is changed in accordance with the instructions from the changing condition in Step S109. The new status of the object X can then be stored in the data memory 1902.

If a condition for a change in the status of the object X is not fulfilled, a poll is taken in Step S104 from the database of change data stored in the data memory 1902 as to whether another changing condition for a change in the status of the object X is fulfilled. This other changing condition can for example be aimed at whether the object X has been moved relative to the previously known location data. If no other changing condition is fulfilled, the method ends in Step S110 without a change in the status of the object X, i.e. the updated status of the object X is identical to the initial status of the object X. If, however, another condition for the change in status is fulfilled, the status of the object X is changed in accordance with the changing condition and/or updated to the corresponding status in Step S109. The new status of the object X can then be stored in the data memory 1902.

If the result in Step S103 is that the location data of the object X is not the same location information as the location data of another object X and/or is not encompassed by the location data of another object X, then a poll is taken in Step S104 from the database of change data stored in the data memory 1902 as to whether another changing condition for a change in the status of the object X is fulfilled. This other changing condition can for example be aimed at whether the object X has been moved relative to the previously known location data. If no other changing condition is fulfilled, the method ends in Step S110 without a change in the status of the object X, i.e. the updated status of the object X is identical to the initial status of the object X. If, however, another condition for the change in status is fulfilled, the status of the object X is changed in accordance with the changing condition and/or updated to the corresponding status in Step S109. The new status of the object X can then be stored in the data memory 1902.

For an object X which is assigned a number of statuses, Steps S104, S106 and S108 can be performed for each individual one of these statuses, wherein it is then possible, for each of the statuses, to check whether a changing condition is fulfilled and/or to compare the status with a corresponding (for example, identical or opposing) status of the other object X. This can for example be performed in a single run of the method according to FIG. 5, as indicated in the drawing.

Computer program elements of the invention may be embodied in hardware and/or software (including firmware, resident software, micro-code, etc.). The computer program elements of the invention may take the form of a computer program product which may be embodied by a computer-usable or computer-readable storage medium comprising computer-usable or computer-readable program instructions, “code” or a “computer program” embodied in said medium for use by or in connection with the instruction executing system. Within the context of this application, a computer-usable or computer-readable medium may be any medium which can contain, store, communicate, propagate or transport the program for use by or in connection with the instruction executing system, apparatus or device. The computer-usable or computer-readable medium may for example be, but is not limited to, an electronic, magnetic, optical, electromagnetic, infrared or semiconductor system, apparatus, device or medium of propagation, such as for example the Internet. The computer-usable or computer-readable medium could even for example be paper or another suitable medium on which the program is printed, since the program could be electronically captured, for example by optically scanning the paper or other suitable medium, and then compiled, interpreted or otherwise processed in a suitable manner. The computer program product and any software and/or hardware described here form the various means for performing the functions of the invention in the example embodiment(s).

Although the invention has been shown and described with respect to one or more particular preferred embodiments, it is clear that equivalent amendments or modifications will occur to the person skilled in the art when reading and interpreting the text and enclosed drawing(s) of this specification. In particular with regard to the various functions performed by the elements (components, assemblies, devices, compositions, etc.) described above, the terms used to describe such elements (including any reference to a “means”) are intended, unless expressly indicated otherwise, to correspond to any element which performs the specified function of the element described, i.e. which is functionally equivalent to it, even if it is not structurally equivalent to the disclosed structure which performs the function in the example embodiment(s) illustrated here. Moreover, while a particular feature of the invention may have been described above with respect to only one or some of the embodiments illustrated, such a feature may also be combined with one or more other features of the other embodiments, in any way such as may be desirable or advantageous for any given application of the invention.

What is claimed is:

1. A medical data method for updating a status of at least one object which relates to a medical applicability of the at least one object, including the following steps:
preparing object data which describes location data and the status of the at least one object;
preparing change data which relates to changes in the status of the at least one object,
wherein
the status of the at least one object is updated on the basis of a comparison between the object data and the change data.

2. The method according to claim 1, wherein the change data describes conditions in which the status of the at least one object changes, wherein on the basis of the change data and object data, a determination is made as to whether a condition for the change in status obtains for the at least one object, and the status which is described by the object data assigned to the at least one object is updated.

3. The method according to claim 1, wherein the change data depends on the location of at least one other object which is different from the at least one object.

4. The method according to claim 1, wherein the change data includes the location of the at least one object and/or time information.

5. The method according to claim 1, wherein the object data is prepared in such a way that a movement of the at least one object can be reconstructed and/or the future movement of the at least one object can be extrapolated on the basis of the object data.

6. The method according to claim 1, wherein the object data additionally includes identification data with respect to the at least one object.

7. The method according to claim 6, wherein the identification data is based on the shape of the at least one object and/or on a pattern of the at least one object and/or includes data with respect to a marker device.

8. The method according to claim 1, wherein information on the update in the status is output on the basis of the comparison between the object data and the change data.

9. The method according to claim 1, wherein when the at least one object is situated at least partially in a patient's body, the object data is based on patient analysis data which is obtained using an imaging method, so as to describe the location of the at least one object in a patient's body.

10. The method according to claim 1, wherein the object data includes target information and actual information on the geometry of the at least one object, wherein the geometry information from the two datasets is compared with each other, and the at least one object is assigned a particular status on the basis of this comparison.

11. The method according to claim 1, wherein a nominal status of the at least one object is compared with the status of the at least one object, whereupon information is transmitted to a management unit on the basis of this comparison.

12. The method according to claim 1, wherein the statuses which the at least one object has passed through are logged, i.e. are stored together with corresponding chronological and spatial information, and/or a particular sequence of statuses is compared with a planned sequence.

13. The method according to claim 1, wherein an initial number of objects and an intermediate number and/or the end number of objects are ascertained, wherein the intermediate number and/or end number is/are compared with the initial number and a notification is output to a user on the basis of the result of this comparison.

14. A program which, when it is running on a computer or is loaded onto a computer, causes the computer to perform a method in accordance with claim 1.

15. A storage medium which comprises a program according to claim 14, or a signal wave which carries information representing the program.

16. A system consisting of: a detection device for detecting the location and identifying an object; and a data processing device for ascertaining the location data and/or identification data of the object.
